(19) United States
(12) Patent Application Publication
(10) Pub. No.: US 2022/0054809 A1
(43) Pub. Date: Feb. 24, 2022

(54) CONTROLLED RELEASE FORMULATIONS IN DELIVERY DEVICES

(71) Applicant: ARGENTA INNOVATION LIMITED, Auckland (NZ)

(72) Inventors: David Anthony GILL, Auckland (NZ); Desmond Ian John MORROW, Auckland (NZ); Michael VENNING, Auckland (NZ)

(21) Appl. No.: 17/275,109

(22) PCT Filed: Sep. 10, 2019
(86) PCT No.: PCT/NZ2019/050120

§ 371 Date: Mar. 10, 2021

(30) Foreign Application Priority Data
Sep. 10, 2018 (NZ) 746215

(51) Int. Cl. A61M 31/00; C08L 39/06; C08L 71/02; C08L 33/02; A61K 45/06; A61K 9/20

(52) U.S. Cl. CPC A61M 31/002; C08L 39/06; C08L 71/02; A61M 2250/00; A61K 45/06; A61K 9/2095; A61M 2205/3334; C08L 33/02

(57) ABSTRACT

The present invention provides an intra-ruminal device comprising a body substantially impervious to rumen fluid, the body comprising a barrel, at least one outlet, and at least one matrix in the barrel, a compression arrangement within the body adapted to bias the column of matrices in the barrel to the at least one outlet, and at least one variable geometry device dependent from the body to assist numen retention, wherein the at least one matrix in the barrel comprises at least one active ingredient and at least one polymer selected from the group consisting of a non-ionic polymer and a cross-linked anionic polymer. The invention also provides a method of making and using the device.

### FIELD OF THE INVENTION

The present invention relates to an intra-ruminal device comprising a formulation that allows for sustained, controlled delivery of one or more active therapeutic or beneficial ingredients to a ruminant animal.

### BACKGROUND TO THE INVENTION

The delivery of pharmaceutically active ingredients or other substances to an animal in a sustained and controlled manner is desirable. Various devices and methods to deliver active ingredients to ruminants are known in the art.

The inability to effectively control the payout rate continues to limit the efficacy of these devices and may lead to problems such as under-dosing and the development of drug resistance.

There is a need for new dosing devices that overcome, avoid or at least partially ameliorate one or more of the aforementioned disadvantages. It is an object of the present invention to go some way to meeting this need; and/or to at least provide the public with a useful choice.

### SUMMARY OF THE INVENTION

In a first aspect the present invention related to an intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device dependent from the body to assist rumen retention,

wherein the at least one matrix in the barrel comprises at least one active ingredient and at least one polymer selected from the group consisting of a non-ionic polymer and a cross-linked anionic polymer.

In a further aspect the present invention related to an intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device dependent from the body to assist rumen retention,

wherein the at least one matrix in the barrel comprises at least one active ingredient and at least one polymer selected from the group consisting of a non-ionic polymer, and a cross-linked anionic polymer, the anionic polymer being a block polymer of polyethylene glycol.

In a further aspect the present invention related to an intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device dependent from the body to assist rumen retention,

wherein the at least one matrix in the barrel comprises at least one active ingredient and at least one polymer selected from the group consisting of a non-ionic polymer, and a cross-linked anionic polymer, the anionic polymer comprising a long chain alkyl acid ester.

In a further aspect the present invention related to an intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device dependent from the body to assist rumen retention,

wherein the at least one matrix in the barrel comprises at least one active ingredient and at least one polymer selected from the group consisting of a non-ionic polyol polymer and a cross-linked anionic polymer.

In a further aspect the present invention related to an intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device dependent from the body to assist rumen retention,

wherein the at least one matrix in the barrel comprises at least one active ingredient and at least one polymer selected from
a) a non-ionic polyol polymer,
b) a cross-linked anionic polymer comprising a long chain alkyl acid ester,
c) a cross-linked anionic polymer being a block polymer of polyethylene glycol,
d) any combination of (a) to (c).

In a further aspect the present invention related to an intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device dependent from the body to assist rumen retention,

wherein the at least one matrix in the barrel comprises at least one active ingredient and at least one polymer selected from a
a) non-ionic polymer comprising
   i) a non-ionic polyol polymer,
   ii) a non-ionic polyethylene oxide homopolymer,
   iii) a non-ionic polyethylene oxide homopolymer having a molecular weight of about 0.9 to about 7 million,
   iv) a non-ionic polyethylene oxide homopolymer having a viscosity of greater than about 7,500 cP,
   v) a non-ionic polyethylene oxide homopolymer that is readily cross-linked; or
b) a cross-linked anionic polymer comprising
   vi) a cross-linked anionic polymer comprising a long chain alkyl acid ester,
   vii) a cross-linked anionic polymer being a block polymer of polyethylene glycol
   viii) an anionic poly (acrylic acid) interpolymer (co-polymer),
   ix) an acrylic acid interpolymer cross-linked with allyl esters of poly alcohols,
   x) an anionic acrylic acid interpolymer that is water insoluble,
   xi) an anionic acrylic acid interpolymer having a viscosity greater than about 45,000 cP,
   xii) a polymer in which the polymerisation process was benzene free,
   xiii) a polymer in which the polymerisation solvent used was a co-solvent comprising ethyl acetate or cyclohexane, or a combination thereof,
   xiv) a non-ionic acrylic acid interpolymer being substantially benzene free,
   xv) a non-ionic acrylic acid interpolymer; or
c) any combination of (i) to (xv).

In some embodiments the non-ionic polymer has any one or more of the following characteristics.
p)
q) a cross-linked anionic polymer comprising a long chain alkyl acid ester,
r) a cross-linked anionic polymer being a block polymer of polyethylene glycol,
s) any combination of (a) to (c).

In a further aspect the invention relates to a method of treating a ruminant animal in need thereof the method comprising administering the intra-ruminal device of any one of the preceding claims to the ruminant animal.

In a further aspect the invention relates to a method of assembling a controlled delivery intra-ruminal comprising
granulating a mixture comprising at least one active ingredient, at least one polymer selected from the group consisting of a non-ionic polymer and a cross-linked anionic polymers, and optionally one or more excipients,
drying the granules,
passing the granules through a sieve, and
tabletting the granules into at least one matrix, and
loading the at least one matrix into the body of an intra-ruminal device.

In a further aspect the invention relates to the use of a controlled release intra-ruminal device to deliver an effective concentration of at least one active ingredient to a ruminant in need thereof.

The following embodiments may relate to any of the above aspects.

Preferably the non-ionic polymer and cross-linked anionic polymer limit interactions with cationic species within the rumen.

Preferably the non-ionic polymer is selected from the group consisting of polyethylene oxide polymers and polyvinylpyrrolidone.

Preferably the polyvinylpyrrolidone has a molecular weight ranging from 35,000 to 60,000 g/molL-1.

Preferably the polyethylene oxide polymer has a molecular weight ranging from 800,000 to 7,250,000 g/molL-1.

Preferably the polyethylene oxide polymer has a viscosity in the range 5,000 to 15,000 cP.

Preferably the cross-linked anionic polymer comprises less than 65% monomers with free anionic groups compared to the total number of monomers.

Preferably the cross-linked anionic polymer is a polyacrylic acid or a polyacrylic acid derivative.

Preferably the cross-linked anionic polymers are cross-linked with allyl sucrose or allylpentaerythritol.

Preferably the cross-linked anionic polymers have a viscosity in the range 40,000 to 67,000 cP.

Preferably the cross-linked anionic polymer comprises a block copolymer of polyethylene glycol and a long chain alkyl acid ester.

Preferably the anionic polymer has an anionic poly (acrylic acid) interpolymer (co-polymer).

Preferably the anionic polymer has an acrylic acid interpolymer cross-linked with allyl esters of poly alcohols.

Preferably the anionic polymer has an anionic acrylic acid interpolymer that is water insoluble.

Preferably the anionic polymer has an anionic acrylic acid interpolymer with a viscosity greater than 45,000 cP.

Preferably the polymerisation process in the formation of the anionic is benzene free.

Preferably during the formation of the anionic polymer the polymerisation solvent is a co-solvent which may include ethyl acetate and cyclohexane.

Preferably the anionic polymer has an non-ionic acrylic acid interpolymer that is substantially (i.e. 95, 96, 97, 98 or 99%) benzene free.

Preferably the anionic polymer has a non-ionic acrylic acid interpolymer that is Carbopol Ultrez 10.

Preferably the non anionic polymer comprises non-ionic polyethylene oxide homopolymers.

Preferably the non anionic polymer comprises non-ionic polyethylene oxide homopolymer(s) with a molecular weight of 0.9, 2, 3, 4, 5, 6, 7, million.

Preferably the non anionic polymer comprises a non-ionic polyethylene oxide homopolymer comprising a viscosity of greater than about 7,500.

Preferably the non anionic polymer comprises a non-ionic polyethylene oxide homopolymer that is readily cross-linked.

Preferably the intra-ruminal device may be for use in a method of treating a ruminant animal.

Preferably the method of treating is independent of pH and ionic effects in the rumen of the ruminant animal.

Preferably the method of treating is independent of pH and ionic effects resulting from changes in the diet of the ruminant animal.

Preferably the at least one active or beneficial ingredient is selected from the group consisting of antibiotics, antifungals, antivirals, steroid hormones, antihistamines, metabolic regulators, for example rumen methane inhibitors/regulators, productivity regulators, corticosteroids, anti-thyroidal agents, parasiticides (ectoparasiticidal agents and/or endoparasiticidal agents), such as for example anthelmintics, non-steroidal anti-inflammatories, nutritional actives, ruminal fermentation modifiers, or a combination thereof. The intra-ruminal device according to any one of the preceding claims, wherein the at least one active ingredient is a parasiticide.

Preferably the at least one active ingredient is a parasiticide.

Preferably the parasiticide is an anthelmintic selected from the group consisting of benzimidazoles, imidazothiazoles, tetrahydropyrimidines, macrocyclic lactones, salicylanides, substituted phenols, aromatic amides, isoquinolines, amino acetonitriles amd spiroindoles, or a combination thereof.

Preferably the method of treating is independent of pH and ionic effects in the rumen of the ruminant animal.

Preferably the method of treating is independent of pH and ionic effects resulting from changes in the diet of the ruminant animal.

Preferably the effective concentration is independent of pH and ionic effects in the rumen of the ruminant animal.

Preferably the effective concentration is independent of pH and ionic effects resulting from changes in the diet of the ruminant animal.

Preferably the ruminant is selected from the group consisting of cattle, goats, sheep and deer.

Other aspects of the invention may become apparent from the following description which is given by way of example only and with reference to the accompanying drawings.

As used herein the term “and/or” means “and” or “or”, or both.

As used herein “(s)” following a noun means the plural and/or singular forms of the noun.

The term “comprising” as used in this specification and claims means “consisting at least in part of”. When interpreting statements in this specification and claims which include that term, the features, prefaced by that term in each statement, all need to be present but other features can also be present. Related terms such as “comprise” and “comprised” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

The entire disclosures of all applications, patents and publications, cited above and below, if any, are hereby incorporated by reference.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.)

### BRIEF DESCRIPTION OF THE DRAWINGS

The invention will now be described by way of example only and with reference to the accompanying figures in which:

FIG. 1 shows the in vitro payout rate per day (as plunger travel in mm/day on the y axis) as a function of time (in days on the x axis) of six replicate capsules (represented by different coloured lines and the codes Z1066, Z1067, Z1068, Z1069, Z1090 and Z1091) comprising cross-linked anionic polymer, Carbomer® 971. The active ingredient in each capsule has been substituted with lactose and sucrose ester. The capsules were tested in a tank comprising water followed by 9 mM, 19 mM and 27 mM calcium solutions respectively, where the change between the solutions is indicated by the dashed vertical lines in the graph. The payout of the capsules was affected by the increased concentration of calcium in solution.

FIG. 2 shows the in vitro payout rate per day (as plunger travel in mm/day on the y axis) as a function of time (in days on the x axis) of 6 replicate capsules (represented by different coloured lines and the codes Z1140, Z1141, Z1142, Z1143, Z1144 and Z1145) comprising a non-ionic polymer, Polyox 301. The capsules were tested in a tank comprising water followed by 9 mM, 19 mM and 27 mM solutions of calcium ions respectively, where the change between the solutions is indicated by the dashed vertical lines in the graph. The payout of the capsules was substantially unaffected by the increased concentration of calcium in solution.

### DETAILED DESCRIPTION OF THE INVENTION

The inventors believe that one of the parameters that may affect the payout rate of intra-ruminal devices known in the art is the concentration of one or more minerals and or ions, for example calcium ions in the rumen of the ruminant.

Different pastures, for example Lucerne compared to Ryegrass, may have different mineral compositions resulting in different amounts of minerals in the rumen of the animal grazing on that particular type of pasture.

Large quantities of certain minerals or ions, for example calcium, in the diet of ruminants may have a negative impact on intra-ruminal devices, for example by limiting the delivery of one or more active ingredients from the device and/or leading to non-linear or unpredictable payout from the device.

The inability to effectively control the payout rate in the face of different concentrations of minerals and/or ions continues to limit the efficacy of intra-ruminal devices known in the art and may lead to problems such as under-dosing and the development of drug resistance.

The present invention broadly relates to an intra-ruminal device for sustained, controlled release of one or more active ingredients to a non-human animal, preferably a ruminant animal.

### 1. Intra-Ruminal Device

The present invention relates to an intra-ruminal device, the device comprising a body substantially impervious to rumen fluid, the body comprising a barrel, at least one outlet, and at least one matrix in the barrel,

a compression arrangement within the body adapted to bias the at least one matrix in the barrel to the at least one outlet, and

at least one variable geometry device dependent from the body to assist rumen retention,

wherein the at least one matrix in the barrel comprises at least one active ingredient and at least one polymer selected from the group consisting of a non-ionic polymer and a cross-linked anionic polymer.

The device of the invention may be used to deliver one or more active therapeutic or beneficial ingredients to a non-human animal.

Preferably the non-human animal may be a ruminant animal, such as for example cattle, goats, sheep, deer, yaks and giraffes, preferably cattle or sheep.

In some embodiments, the body of the intra-ruminal device may be rigid and hold its shape when the at least one matrix comprising the one or more active ingredients is inserted into the barrel of the device, and when the device is administered to an animal.

The body of the intra-ruminal device may be formed into a number of suitable shapes. Preferably the body of the intra-ruminal device is cylindrically shaped, and preferably the cross section of the body is circular. Preferably one end of the body may taper in to a reduced diameter to aid the passage of the intra-ruminal device down the oesophagus to the rumen.

The diameter of the body of the intra-ruminal device is small enough to pass down the oesophagus of a ruminant animal with ease and large enough to accommodate at least one matrix in the barrel. The diameter of the barrel depends on, for example the thickness of the body of the intra-ruminal device. In some embodiments the diameter of the intra-ruminal device and the diameter of the barrel may be very similar, the difference being the result of the thickness of the body.

In some embodiments the diameter of the intra-ruminal device may be less than about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, or 4.5 cm, and useful ranges may be selected from any of these values (for example the diameter of the intra-ruminal device may be from about 1 to about 4.5, about 1 to about 4 cm, from about 1 to about 3.5 cm, about 1 to about 3, about 1 to about 2.5, about 1 to about 2, about 1 to about 1.5, about 1.2 to about 4.5, about 1.2 to about 4, about 1.2 to about 3.5, about 1.2 to about 3, about 1.2 to about 2.5, about 1.2 to about 2, about 1.2 to about 1.5, about 1.5 to about 4.5, about 1.5 to about 3.5, about 1.5 to about 3, about 1.5 to about 2.5, or about 1.5 to about 2).

The length of the body of the intra-ruminal device is short enough not to impede progress along the oesophagus to the reticulo-rumen.

The length of the body of the device can vary to, for example, accommodate more or fewer matrices. The length of the body may also vary depending on, for example, the target species to which the intra-ruminal device is to be administered the size of the animal, the dose and pay-out period.

The length of the body may be from about 40, 45, 50, 55, 60, 65, 70, 75, 80, 85, 90, 95, 100, 105, 110, 115, 120, 125, 130, 135, 140, 145, 150, 155, 160, 165, 170, 175 or 180 mm or more, and useful ranges may be selected from any of these values (for example from about 40 mm to about 180 mm, 40 mm to about 150 mm, about 40 mm to about 120 mm, about 40 mm to about 100 mm, about 40 mm to about 75 mm, about 70 mm to about 180 mm, about 70 mm to about 160 mm, about 70 mm to about 160 mm, about 70 mm to about 140 mm, about 70 mm to about 120 mm, about 70 mm to about 100 mm, about 75 mm to about 180 mm, about 75 mm to about 165 mm, about 75 mm to about 145 mm, about 75 mm to about 125 mm, or about 75 mm to about 105 mm). For example in some embodiments the length of the body of an intra-ruminal device to be administered to sheep and other small ruminants may be from about 76 mm to about 90 mm, and the length of the body of the intra-ruminal device to be administered to cattle and other similar-sized ruminants may be from about 97 to about 170 mm.

In some embodiments, the body of the intra-ruminal device may be impervious to intra-ruminal fluid but may allow permeation of gases. In some instances the permeability of the wall of the intra-ruminal device may require additional features to improve permeability of gasses and to prevent the formation of a partial vacuum above the compression arrangement that may affect smooth operation of the biasing system. The additional feature may further include an aperture above the starting position of the compression arrangement that increases gas permeation but prevents or substantially prevents the ingress of ruminal fluids. This part of the intra-ruminal device may include an area of modified polymer or a vent incorporating a membrane such as a semi-permeable membrane. In various embodiments the body of the intra-ruminal device may be made from a pharmaceutical grade polymer or co-polymer. Suitable polymers and co-polymers will be apparent to a person skilled in the art.

The intra-ruminal device comprises a retention means that serves to keep the device in the rumen and to prevent regurgitation. This may be achieved in a number of ways. For example the retention means may comprise a weighted component or part. The weighted component may be for example an area of the body that is made of a material of higher density than the material used to make the rest of the body. The weighted component may therefore ensure that the intra-ruminal device remains at the bottom of the rumen cavity to avoid regurgitation.

In various embodiments, the retention means may comprise a variable geometry device, preferably a retractable wing or pair of wings, preferably on one end of the body. The variable geometry device, preferably the wings are pressed against the side of the body when administered and spring out after administration to prevent regurgitation. In some embodiments the intra-ruminal device may comprise more than one retention means, for example a variable geometry device such as a wing or pair of wings and one or more weighted components.

The variable geometry device, for example wings, may be pressed against the side of the body using a number of means. For example, water soluble tape or adhesive may be used to hold the wings against the body.

In some embodiments the variable geometry device, for example wings, may be pressed against the side of the body by an applicator during dosing.

In some embodiments the variable geometry device, for example wings may be pressed against the side of the body using a pharmaceutical grade polymer or co-polymer that is readily dissolved by the contents of the rumen or using a polymer or co-polymer that melts at the temperature of the rumen, for example a polymer that melts at a temperature of from about 37.5, 38, 39, 39.5, 40, 40.5 or 41° C., and useful ranges may be selected from any of these values (for example from about 39 to about 40° C., or from about 38 to about 41° C.). Preferably the melting point of the polymer or co-polymer is from about 38.5 to about 40.5° C. to avoid the polymer melting in the oesophagus of the ruminant and releasing the wings from the side of the body before the device enters the rumen.

In some embodiments the variable geometry device, for example wings may be made from the same polymeric material as the body, or they may be made from a different polymeric material. In various embodiments the variable geometry device, for example wings may be made of a polymeric material that is less rigid than the polymer used to make the body, to allow the wings to be retained against the side of the body during administration to an animal. Suitable polymeric materials will be apparent to a person skilled in the art and may include for example any pharmaceutical grade polymers that are sufficiently pliable to be held against the side of the intra-ruminal device when administered. In various embodiments the wings or part of the wings may be made of polypropylene or a co-polymer thereof.

In some embodiments, the body, and the variable geometry device, for example the wing(s) of the intra-ruminal device may be manufactured from one or more parts moulded from plastic materials (e.g. polypropylene) and may be fabricated together by adhesive and/or welding.

The intra-ruminal device comprises a compression arrangement located in the barrel of the device to compress the composition containing the active ingredient(s) towards the at least one outlet for release to the rumen. In various embodiments the at least one outlet is located at one end of the body and the compression arrangement biases the at least one matrix in the barrel of the intra-ruminal device towards the at least one outlet. The force exerted by the compression arrangement is intended to exceed any frictional forces generated between the core and the internal wall of the device over the entire distance that the compression arrangement travels to ensure consistent and linear delivery of the at least one matrix.

In some embodiments, the compression arrangement may comprise a plunger and biasing means. In various embodiments the biasing means may be a spring.

In some embodiments, the biasing means, such as a spring, may be made of materials such as alloys of steel, for example stainless steel, carbon steel, oil tempered wire, chrome silicon steel or chrome vanadium steel. Other alloys may also be used, for example Inconel, Monel, beryllium, copper or phosphor bronze. Other suitable materials will be apparent to those skilled in the art.

In various embodiments the compression arrangement may be adapted to be extendible to at least about 45, 50, 55, 60, 65, 70, 75, 80, 85, 90, 95 or 100% of the length of the body, and suitable ranges may be selected from any of these values (for example from about 45% to about 100%, about 45 to about 75%, from about 45 to about 60%, about 50% to about 100%, about 60 to about 80%, about 50 to about 80%, about 50 to about 60%, about 60 to about 100%, about 60 to about 80%, about 70 to about 100%, about 70 to about 80%, or from about 80% to about 100%).

In exemplary embodiments the compression arrangement may comprise a spring that is adapted to push a plunger to extend the compression arrangement to at least about 80, 85, 90, 95 or 100% of the length of the body.

In various embodiments the pressure exerted by the compression arrangement, for example the biasing means such as a spring, may remain substantially constant for the entire payout period, the substantially constant pressure leading to a linear or substantially linear (>0.95) sustained delivery of one or more active ingredients as described herein.

Without wishing to be bound by theory the inventors believe the pressure exerted by the compression arrangement, that is, the pressure biasing the at least one matrix towards the at least one outlet contributes to control of the payout period.

In various embodiments the barrel of the intra-ruminal device comprises at least one matrix comprising at least one active ingredient.

In some embodiments the barrel of the intra-ruminal device may comprise one matrix only or more than one matrix, for example 1, 2, 3, 4, 5, 6, 7, 8. 9, 10, 11, 12, 13, 14, 15, 16, 17, 18, 19, 20, 21, 22, 23, 24, 25, 26, 27, 28, 29, 30 or more matrices, and useful ranges may be selected from any of these values, for example 1 to 30 matrices, 1 to 25, 1 to 20 matrices, 1 to 15, 1 to 10 matrices, 1 to 5, 5 to 30, 5 to 25, 5 to 20, or 5 to 15 matrices. The at least one matrix may be any shape adapted to fit inside the barrel of the device. In various embodiments the barrel of the intra-ruminal device may comprise more than one matrix. The form of the at least one matrix may be for example a tablet, a capsule, a caplet or a wafer.

In some embodiments the at least one matrix may be shaped to allow them to be stacked along the longitudinal axis of the body of the intra-ruminal device, such that they are sequentially presentable to the rumen, as originally proposed in the Laby device. Preferably the at least one matrix is a tablet, preferably disc-shaped.

In some embodiments the diameter of the at least one matrix may be less than about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mm, and useful ranges may be selected from any of these values (for example the diameter of the matrices may be from about 9 to about 40 mm about 9 to about 30 mm, about 9 to about 20 mm, about 9 to about 10 mm, about 10 to about 35 mm, about 10 to about 25 mm, about 10 to about 15 mm, form about 11 to about 40, 11 to about 38, 11 to about 36, 11 to about 34, 11 to about 32 mm, 11 to about 30 mm, 11 to about 28 mm, 11 to about 26 mm, 11 to about 24 mm, 11 to about 22 mm, 11 to about 20 mm, 11 to about 19 mm, 11 to about 18 mm, 11 to about 17 mm, 11 to about 16 mm, 11 to about 15 mm, 11 to about 14 mm, 12 to about 40 mm, 12 to about 38, 12 to about 36, 11 to about 34, 12 to about 32 mm, 12 to about 30 mm, 12 to about 28 mm, 12 to about 26 mm, 12 to about 24 mm, 12 to about 22 mm, 12 to about 20 mm). For example in some embodiments the at least one matrix for use in intra-ruminal devices to be administered to sheep may be from about 11 to about 15 mm in diameter and the at least one matrix for use in intra-ruminal devices to be administered to cows may be from about 15 to about 32 mm in diameter.

The diameter of the at least one matrix comprising the one or more active ingredients must be such that the diameter is small enough to fit into the barrel of the device. For example if the diameter of the barrel of the device is 30 mm, then the matrix may have a diameter of for example around 29.5 mm. In various embodiments the diameter of the matrix may be sufficiently close to the internal diameter of the barrel to substantially prevent ingress of rumen fluid between the core and barrel without preventing movement of the matrix within the barrel.

In some embodiments the device may comprise a plurality of matrices, for example a plurality of compressed tablets, the number of matrices depending on the length of the body of the device, the thickness of the matrix, the desired payout period and the amount of active present in the at least one matrix. For example in some embodiments the thickness of the at least one matrix may be from at least about 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75 or 10 mm or more, and useful ranges may be selected between any of these values, for example from about 1 to about 10 mm, about 1 to about 8 mm, about 1 to about 6 mm, about 1 to about 4 mm, about 2 to about 10 mm, about 2 to about 8 mm, about 2 to about 6 mm, about 2 to about 4 mm, about 3 to about 10 mm, about 3 to about 9 mm, about 3 to about 7 mm, about 3 to about 5 mm, about 5 to about 10 mm, about 5 to about 9 mm, about 5 to about 8 mm, about 5 to about 7 mm, about 7 to about 10 mm, or from about 7 to about 9 mm.

In some embodiments the barrel may comprise one matrix only, for example one solid core comprising at least one active ingredient and optionally one or more excipients. In such embodiments the matrix may substantially span the length of the barrel from the compression arrangement to the end of the body comprising the at least one outlet.

In some embodiments the solid core may be continuous or constructed of individual compressed matrices or units (tablets) arranged in a stack.

In some embodiments, the active ingredient(s) may be released in to the rumen in a controlled manner by contact of the matrix comprising the active ingredient(s) with the intra-ruminal fluid allowing erosion or dissolution of the matrix in to the rumen.

In some embodiments, a seal exists between the rumen-facing end of the matrix comprising the active ingredient(s) and the barrel of the intra-ruminal device. Without wishing to be bound by theory the inventors believe that an ineffective seal between the barrel and rumen-facing end of the matrix comprising the active ingredient(s) may allow other surfaces of the matrix or other matrices in the stack to swell, adversely affecting, or stopping reliable payout of the one or more active ingredients.

In various embodiments, the at least one outlet may be located at one end of the body. The outlet may be from about 1, 2, 3, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22 mm or more in diameter, and useful ranges may be selected in between any of these values (for example from about 1 to about 22 mm, about 1 to about 20 mm, about 1 to about 18.5 mm, about 1 to about 15 mm, about 1 to about 12 mm, about 1 to about 10 mm, about 1 to about 5 mm, about 3 to about 22 mm, about 3 to about 20 mm, about 3 to about 18.5 mm, about 3 to about 15 mm, about 3 to about 12 mm, about 4 to about 22 mm, about 4 to about 20 mm, about 4 to about 18.5 mm in diameter, about 4 to about 15 mm, or about 4 to about 12 mm). It will be understood by a person skilled in the art that the size of the outlets will depend on factors such as for example, the intended payout rate.

In various embodiments the diameter of the at least one outlet may be selected to ensure that a sufficient ridge exists to seal against the at least one matrix in the barrel of the intra-ruminal device.

In various embodiments, the intra-ruminal device may comprise an end cap provided to one end of the body.

In various embodiments, an end cap is provided to the end of the body comprising the at least one outlet.

In various embodiments the end cap comprises the at least one outlet.

In various embodiments the end cap may be permanently fixed to the body of the intra-ruminal device, for example may be integral with the body of the intra-ruminal device.

In various embodiments the end cap may be removably attached to the body of the intra-ruminal device.

In some embodiments the end cap may comprise one outlet only.

In some embodiments the end cap may comprise more than one outlet, for example 2, 3, 4, 5, 6, 7, 8, 9, 10 or more outlets and useful ranges may be selected from any of these values, for example 2 to 6 outlets or 3 to 8 outlets. Preferably the one or more outlets are located at or near the centre of the end cap. Preferably if the end cap comprises multiple outlets then the outlets are substantially equidistant from one another.

In some embodiments, the end cap may be made of the same material as the body or a different material. In various embodiments the end cap is made of a polymeric material that is stable under the conditions present in the rumen of the animal.

The end cap, when present, may be secured to the end of the body by any suitable means. For example, the end cap may be welded or glued to the end of the barrel, preferably welded.

### 2. Matrix Contents

The intra-ruminal device of the invention comprises at least one matrix. The at least one matrix may comprise one or more active ingredients, one or more polymers and excipients in a ratio that allows for the delivery of a therapeutically effective amount of the one or more active ingredients to the non-human animal, preferably ruminant.

### 2.1. Polymer

The inventors have advantageously discovered that the rate of release and/or linearity of release of nutritional or pharmaceutically active ingredient(s) from at least one matrix in an intra-ruminal device may be modulated or controlled using one or more polymers in the matrices, preferably one or more polymers selected from the group consisting of non-ionic and cross-linked anionic polymers.

A non-ionic polymer is a polymer that does not comprise any charged groups. A cross-linked anionic polymer is a polymer that comprises negatively charged (anionic groups), for example carboxylate groups and comprises one or more linkages between different polymer chains. The linkages may be covalent or ionic bonds and the cross-linked anionic polymer may be a natural or synthetic polymer.

Without wishing to be restricted by theory the non-ionic polymer and cross-linked anionic polymer limit interactions with cationic species within the rumen which may affect the critical attributes of the polymer(s).

In various embodiments the polymers, preferably non-ionic polymers or cross-linked anionic polymers for use in the matrices of the intra-ruminal devices are polymers that form strong gels and/or have a low swelling capacity.

The strength of a gel may be determined in a number of ways, for example using the Bloom test which involves determining the weight in grams required to depress the surface of a gel at a specified temperature by 4 mm using a plunger with a diameter of 0.5 inches.

In some embodiments the non-ionic polymers and the cross-linked anionic polymers in the matrix and the intra-ruminal device of the invention may give rise to a Bloom strength in the range of from about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450. 460, 470, 480, 490 or 500 or more and useful ranges may be selected from any of these values (for example from about 1 to about 500, 10 to about 500, 100 to about 500, 200 to about 500, about 300 to about 500, about 400 to about 500, about 50 to about 450, about 50 to about 350, about 50 to about 350 or about 50 to about 250).

The swelling capacity of a polymer may be calculated in a number of ways. For example the swelling capacity of a polymer may be calculated by taking an amount (for example 0.1 g) of the polymer and exposing it to 100 mL of deionised water. After 20 minutes the polymer is weighed and the percentage weight change is calculated according to formula 1 below:

((Final weight (after swelling)−Initial weight (before swelling)/Initial weight (before swelling))×100%.

In some embodiments the swelling capacity of the polymer may be from about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 180, 185, 190, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500% or more as calculated using formula 1, and useful ranges may be selected from any of these values (for example the swelling capacity may be from about 1 to about 500%, 5 to about 500%, 10 to about 500%, 100 to about 500%, 200 to about 500%, 300 to about 500%, 400 to about 500%, 1 to about 400%, 1 to about 300% 1 to about 200%, 1 to about 100%, 1 to about 90%, 1 to about 75%, 1 to about 50%, 1 to about 25%, or from about 1% to about 10%). In some embodiments, the polymer, such as the non-ionic or cross-linked anionic polymer is present in an amount of from about 0.5, 1, 2, 3, 4, 5, 6, 7, 8, 9, 10, 12.5, 15, 17.5, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75 80, 85, or 90% by weight of the matrix, and useful ranges may be selected from any of these values (for example from about 1 to about 50% by weight or from about 2 to about 45% by weight of the matrix, preferably from about 0.5 to about 90, about 0.5 to about 70, about 0.5 to about 50, about 0.5 to about 30, about 0.5 to about 10, about 0.5 to about 8, about 0.5 to about 6, about 1 to about 90, about 1 to about 60, about 1 to about 40, about 1 to about 10, about 1 to about 8, about 1 to about 6, about 1 to about 4, about 2 to about 90, about 2 to about 70, about 2 to about 50, about 2 to about 30, about 2 to about 10, about 2 to about 8, or about 2 to about 6).

In some embodiments the non-ionic polymers may be homopolymers or they may be co-polymers comprising two or more polymers, each of which is non-ionic, preferably they are homopolymers. In some embodiments the non-ionic polymers may be alternating, branched or block co-polymers.

In various embodiments the non-ionic polymers may be homopolymers comprising an acyclic polymer backbone.

In some embodiments the non-ionic polymers may be cross-linked.

In various embodiments the polymer is a cross-linked anionic polymer comprising a long chain alkyl acid ester.

In various embodiments the polymer is a cross-linked anionic polymer being a block polymer of polyethylene glycol.

In various embodiments the non-ionic polymers with an acyclic polymer backbone may be polyvinylpyrrolidone or polyethylene oxide.

In exemplary embodiments the non-ionic polymer may be polyvinylpyrrolidone with a molecular weight ranging from about 35,000, 36,000, 37,000, 38,000, 39,000, 40,000, 41,000, 42,000, 43,000, 44,000, 45,000, 46,000, 47,000, 48,000, 49,000, 50,000, 51,000, 52,000, 53,000, 54,000, 55,000, 56,000, 37,000, 58,000, 59,000 or 60,000 g/mol-1, and suitable ranges may be selected from any of these values (for example from about 35,000 to about 60,000, about 35,000 to about 50,000, about 35,000 to about 40,000, about 40,000 to about 60,000, about 40,000 to about 50,000, about 42,000 to about 60,000, about 42,000 to about 50,000, about 44,000 to about 60,000, about 44,000 to about 58,000, about 44,000 to about 65,000, about 44,000 to about 54,000, about 44,000 to about 52,000, about 44,000 to about 50,000, about 42,000 to about 60,000, about 42,000 to about 58,000, about 42,000 to about 56,000, about 44,000 to about 54,000, about 42,000 to about 52,000, or about 42,000 to about 50,0000, about 35,000 to about 60,000, about 35,000 to about 55,000, about 35,000 to about 50,000, or from about 35,000 to about 45,000 g/mol-1).

In exemplary embodiments, the non-ionic polymer may be polyvinylpyrrolidone comprising from about 310, 320, 330, 340, 350, 360, 370, 380, 385, 390, 395, 400, 405, 410, 415, 420, 425, 430, 435, 440, 445, 450, 455, 460, 465, 470, 475, 480, 485, 490, 495, 500, 505, 510, 515, 520, 530, 535 or 540 monomer units and useful ranges may be selected from any of these values (for example from about 540, 310 to about 500, about 310 to about 400, about 350 to about 540, about 350 to about 500, about 350 to about 400, about 380 to about 540, about 380 to about 500, about 380 to about 400, about 395 to about 540, about 395 to about 500, about 395 to about 485, about 395 to about 450, about 395 to about 430, about 395 to about 400 monomer units).

In some embodiments the anionic polymer has any one or more of the following characteristics.
An anionic poly (acrylic acid) interpolymer (co-polymer).
An acrylic acid interpolymer cross-linked with allyl esters of poly alcohols.
An anionic acrylic acid interpolymer is water insoluble.
An anionic acrylic acid interpolymer having a viscosity of greater than about 45,000 cP.
Formed in which the polymerisation process is benzene free.
Formed in which the polymerisation solvent is a co-solvent which includes ethyl acetate and cyclohexane.
The anionic acrylic acid interpolymer is essentially benzene free.
The anionic acrylic acid interpolymer is Carbopol Ultrez 10.

In some embodiments the non-ionic polymer has any one or more of the following characteristics.
A non-ionic polyethylene oxide homopolymers.
The non-ionic polyethylene oxide homopolymer has a molecular weight of about 0.9 to about 7 million.
The non-ionic polyethylene oxide homopolymer has a viscosity of greater than about 7,500.
The non-ionic polyethylene oxide homopolymer is readily cross-linked.

In various embodiments the polymer is a non-ionic polyol polymer.

In various embodiments the non-ionic polymer is a polyethylene oxide polymer, for example a polymer marketed under the names POLYOX™ 301, POLYOX™ 303 or POLYOX™ 1105.

In various embodiments the polyethylene oxide polymer may have a molecular weight of from about 800,000, 850,000, 900,000, 950,000, 1,000,000, 1,250,000, 1,500,000, 1,750,000, 2,000,000, 2,250,000, 2,500,000, 2,750,000, 3,000,000, 3,250,000, 3,500,000, 3,750,000, 4,000,000, 4,250,000, 4,500,000, 4,750,000, 5,000,000, 5,250,000, 5,500,000, 5,750,000, 6,000,000, 6,250,000, 6,500,000, 6,750,000, 7,000,000 or 7,250,000 g/mol-1, and useful ranges may be selected from any of these values (for example a molecular weight of from about 800,000 to 7,000,000 about 800,000 to about 6,000,000, about 800,000 to about 5,000,000, about 800,000 to about 4,000,000, about 800,000 to about 3,000,000, about 800,000 to about 2,000,000, about 900,000 to about 7,000,000, m about 900,000 to about 6,000,000, about 900,000 to about 5,000,000, about 900,000 to about 4,000,000, about 900 to about 3,000,000, about 900,000 to about 2,000,000, about 1,000,000 to about 5,000,000, about 1,000,000 to about 4,000,000, about 1,000,000 to about 3,000,000, of from about 1,000,000 to about 2,000,000 gmol-1). In exemplary embodiments, the non-ionic polymer may be a polyethylene oxide polymer, comprising from about 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000 or 165,000 monomer units and useful ranges may be selected from any of these values (for example from about 15,000 to about 165,000, about 15,000 to about 150,000, about 15,000 to about 10,000, about 15,000 to about 75,000, about 15,000 to about 50,000, about 20,000 to about 165,000, about 20,000 to about 155,000, about 20,000 to about 100,000, about 20,000 to about 80,000, about 20,000 to about 60,000, or from about 20,000 to about 40,000 monomer units).

In various embodiments the non-ionic polymers, preferably non-ionic polymers comprising an acyclic backbone, may have a viscosity of from about 5,000, 5,250, 5,500, 5,750, 6,000, 6,250, 6,500, 6,750, 7,000, 7,250, 7,500, 7,750, 8,000, 8,250, 8,500, 8,750, 9,000, 9,250, 9,500, 9,750, 10,000, 10,250, 10,500, 10,750, 11,000, 11,250, 11,500, 11,750, 12,000, 12,250, 12,500, 12,750, 13,000, 13,250, 13,500, 13,750, 14,000, 14,250, 14,500, 14,750, or 15,000 cP (0.5 wt % at pH 7.5), and useful ranges may be selected from these values (for example a viscosity of from about 5,000 to about 15,000, about 5,000 to about 12,000, about 5,000 to about 10,000, about 7,500 to about 15,000 cP or from about 7,500 to about 10,000 cP).

In some embodiments the cross-linked anionic polymers may comprise anionic groups such as for example, carboxylates, sulfates, sulfonates and phosphates, preferably carboxylates such as acrylates.

In various embodiments the cross-linked anionic polymers may be modified polyacrylic acid polymers with a high density of crosslinks that form strong gels.

In some embodiments the cross-linked anionic polymers may comprise a percentage of monomers with free anionic groups, such as free acid groups, as a percentage of the total number of monomers of less than about 50, 51, 52, 53, 54, 55, 56, 57, 58, 59, 60, 61, 62, 63, 64, or 65%, and useful ranges may be selected from any of these values (for example from about 50 to about 65, about 50 to about 60, about 50 to about 59, about 50 to about 58, about 50 to about 57, about 50 to about 56, about 50 to about 55, about 50 to about 54, about 50 to about 53, about 50 to about 52, about 50 to about 51, about 55 to about 65, about 55 to about 64, about 55 to about 63, about 55 to about 62, about 55 to about 61, about 55 to about 60, about 55 to about 59, about 55 to about 58, about 55 to about 57, about 55 to about 56, about 56 to about 65, about 56 to about 64, about 56 to about 63, about 56 to about 62, about 56 to about 61, about 56 to about 60, about 56 to about 59, about 56 to about 58, about 56 to about 57, about 57 to about 65, about 57 to about 64, about 57 to about 63, about 57 to about 62, about 57 to about 62, about 57 to about 61, or about 57 to about 60%).

The cross-linked polymers may be cross-linked with, for example, allyl sucrose or allylpentaerythritol.

In various embodiments the cross-linked anionic polymers, preferably cross-linked polyacrylic acid polymers, may have a viscosity of from about 40,000, 40,250, 40,500, 40,750, 41,000, 41,250, 41,500, 41,750, 42,000, 42,250, 52,500, 42,750, 43,000, 43,250, 43,500, 43,750, 44,000, 44,250, 44,500, 44,750, 45,000, 45,250, 45,500, 45,750, 46,000, 46,250, 46,500, 46,750, 47,000, 47,250, 47,500, 47,750, 48,000, 48,250, 48,500, 48,750, 49,000, 49,250, 49,500, 49,750, 50,000, 51,000, 51,250, 51,500, 51,750, 52,000, 52,250, 52,500, 52,750, 53,000, 53,250, 53,500, 53,750, 54,000, 54,250, 54,500, 54,750, 55,000, 55,250, 55,500, 55,750, 56,000, 56,250, 56,500, 56,750, 57,000, 57,250, 57,500, 57,750, 58,000, 58,250, 58,500, 58,750, 60,000, 60,250, 60,500, 60,750, 61,000, 61,250, 61,500, 61,750, 62,000, 62,250, 62,500, 62,750, 63,000, 63,250, 63,500, 63,750, 64,000, 64,250, 64,500, 64,750, 65,000, 65,125, 65,250, 65,500, 65,750, 66,000, 66,250, 66,500, 66,750, or 67,000 cP, and useful ranges may be selected from these values (for example a viscosity of from about 45,000 to about 60,000 cP).

In various embodiments the cross-linked anionic polymers may be high crosslink density polymers such as for example the polymer marketed under the name Carbopol® Ultrez 10NF.

### 2.2. Active Ingredients

The at least one matrix in the intra-ruminal devices of the present invention delivers a therapeutic quantity of one or more active ingredients. The active ingredient(s) are delivered from the intra-ruminal device and may have a local action, for example in the gastrointestinal tract, and/or may have activity within the rumen including the microbial or enzymic environment, and/or they may be absorbed in to the systematic circulation to impart a therapeutic response in other body compartments including for example major organs and tissues.

A wide range of active ingredients may be delivered from the at least one matrix in the intra-ruminal devices of the present invention.

The intra-ruminal device of the invention comprises at least one matrix, the at least one matrix defining a core. In some embodiments the core may comprise a single therapeutic or a combination of blended therapeutics. In some embodiments the therapeutics may be separated throughout the core using individual matrices.

In some embodiments the at least one matrix may comprise one or more antibiotics, antifungals, antivirals, steroid hormones, antihistamines, metabolic regulators, for example rumen methane inhibitors/regulators, productivity regulators, corticosteroids, anti-thyroidal agents, parasiticides (ectoparasiticidal agents and/or endoparasiticidal agents), such as for example anthelmintics, non-steroidal anti-inflammatories, nutritional actives, ruminal fermentation modifiers, or a combination thereof. In some embodiments the at least one matrix may comprise one or more vitamins, for example vitamin A, vitamin E, vitamin B₁₂, vitamin B₃, d-pantothenic acid (vitamin B₅), folic acid, vitamin B₆, vitamin B₁, vitamin D₃, vitamin C, vitamin B₂ vitamin B_{y} or H. As another example, the nutritional active could be a pro-vitamin, for example beta-carotene or panthenol.

In some embodiments the nutritional active may be an amino acid. Suitable amino acids include but are not limited to the 20 naturally occurring L-amino acids, for example arginine, isoleucine, leucine, lysine, etc.

In some embodiments the nutritional active may be a co-enzyme, for example co-enzyme Q.

In some embodiments the nutritional active may be a mineral. Non-limiting examples of minerals include potassium, sodium, manganese, zinc, iron, calcium, copper, cobalt, iodine, chlorine and selenium. In some embodiments the mineral may be in the form of a suitable salt.

In some embodiments the at least one matrix may comprise one or more anti-microbial ingredients for example antibiotics, antifungals, antivirals, anthelmintics, and the like.

Suitable antibiotic agents may be those that act as inhibitors of cell wall synthesis (e.g. penicillins, cephalosporins, bacitracin and vancomycin), inhibitors of protein synthesis (aminoglycosides, macrolides, lincosamides, streptogramins, chloramphenicol, tetracyclines), inhibitors of membrane function (e.g. polymixin B and colistin), inhibitors of nucleic acid synthesis (e.g. quinolones, metronidazole, and rifampin), or inhibitors of other metabolic processes (e.g. anti-metabolites, sulfonamides, and trimethoprim). Non-limiting examples of antibiotics include polyethers, ionophores such as monensin and salinomycin, beta-lactams such as penicillins, aminopenicillins (e.g., amoxicillin, ampicillin, hetacillin, etc.), penicillinase resistant antibiotics (e.g., cloxacillin, dicloxacillin, methicillin, nafcillin, oxacillin, etc.), extended spectrum antibiotics (e.g., axlocillin, carbenicillin, mezlocillin, piperacillin, ticarcillin, etc.); cephalosporins (e.g., cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, etc.); monobactams such as aztreonam; carbapenems such as imipenem and eropenem; quinolones (e.g., ciprofloxacin, enrofloxacin, difloxacin, orbifloxacin, marbofloxacin, etc.); chloramphenicols (e.g., chloramphenicol, thiamphenicol, florfenicol, etc.); tetracyclines (e.g., chlortetracycline, tetracycline, oxytetracycline, doxycycline, minocycline, etc.); macrolides (e.g., erythromycin, tylosin, tlimicosin, clarithromycin, azithromycin, etc.); lincosamides (e.g., lincomycin, clindamycin, etc.); aminoglycosides (e.g., gentamicin, amikacin, kanamycin, apramycin, tobramycin, neomycin, dihydrostreptomycin, paromomycin, etc.); sulfonamides (e.g., sulfadmethoxine, sfulfamethazine, sulfaquinoxaline, sulfamerazine, sulfathiazole, sulfasalazine, sulfadiazine, sulfabromomethazine, suflaethoxypyridazine, etc.); glycopeptides (e.g., vancomycin, teicoplanin, ramoplanin, and decaplanin; and other antibiotics (e.g., rifampin, nitrofuran, virginiamycin, polymyxins, tobramycin, etc.).

In some embodiments the at least one matrix may comprise one or more antifungal active ingredients for example one or more polyenes, azoles, allylamines, morpholines, antimetabolites, and combinations thereof. For example in some embodiments the at least one matrix may comprise one or more of fluconazole, itraconazole, clotrimazole, ketoconazole, terbinafine, 5-fluorocytosine, and amphotericin B, or combinations thereof.

Non-limiting examples of antivirals that may be present in the at least one matrix may include didanosine, lamivudine, stavudine, zidovudine, indinavir, and ritonavir.

In some embodiments the at least one matrix may comprise one or more steroid hormone, for example steroid hormones such as growth promoters and production enhancers. In some embodiments, the steroid hormone may be natural steroid hormone, such as for example estradiol, progesterone, and testosterone, or a synthetic steroid hormone, such as trenbolone acetate, estradiol benzoate, estradiol 170, and melengestrol acetate, and/or zeranol.

Steroid hormones that may be present in at least one matrix may comprise for example natural and synthetic steroid hormones, steroid hormone precursors, steroid hormone metabolites, and derivatives thereof that are structurally derived from cholesterol. Steroid hormones may be synthesized from cholesterol via pathways that involve cytochrome P450 (cP450) enzymes, which are heme-containing proteins.

In some embodiments the at least one matrix may comprise one or more steroid hormones such as for example androgens, estrogens, progestogens, mineral corticoids, and glucocorticoids. Exemplary androgens include, but are not limited to, testosterone, dehydroepiandrosterone, dehydroepiandrosterone sulphate, dihydrotestosterone, androstenedione, androstenediol, androstanedione, androstanediol, and any combination thereof. Exemplary estrogens include, but are not limited to, estrone, estradiol, estriol, estetrol, equilin, equilenin, and any combination thereof. Exemplary progestogens include, but are not limited to, progesterone, 17-hydroxy-progesterone, pregnenolone, dihydroprogesterone, allopregnanolone, 17-hydroxy-pregnenolone, 17-hydroxy-dihydroprogesterone, 17-hydroxy-allopregnanolone, and any combination thereof. Exemplary mineralcorticoids include, but are not limited to, aldosterone, 11-deoxycorticosterone, fludrocortisones, 11-deoxy-cortisol, pregnenedione, and any combination thereof. Exemplary glucocorticoids, include, but are not limited to, cortisol (hydrocortisone), corticosterone, 18-hydroxy-corticosterone, cortisone, and any combination thereof.

In some embodiments the at least one matrix may comprise one or more anti-histamines, such as for example clemastine, clemastine fumarate (2(R)-[2-[1-(4-chlorophenyl)-1-phenyl-ethoxy]ethyl-1-methylpyrrolidine), dexmedetomidine, doxylamine, loratidine, desloratidine and promethazine, and diphenhydramine, or pharmaceutically acceptable salts, solvates or esters thereof.

In some embodiments the at least one matrix may comprise one or more active ingredients that are adapted to modify intra-ruminal fermentation processes.

In some embodiments the at least one matrix may comprise one or more metabolic regulators, such as for example one or more methane inhibitors/regulators, or fermentation regulators/modifiers.

In some embodiments the at least one matrix may comprise one or more productivity regulators, for example polyethers such as monensin. In some embodiments, the productivity regulator may be a productivity enhancer.

In exemplary embodiments the at least one matrix may comprise one or more anthelmintic agents, for example one or more benzimidazoles, imidazothiazoles, tetrahydropyrimidines, macrocyclic lactones, salicylanilides, substituted phenols, aromatic amides, isoquinolines, amino acetonitriles, spiroindoles, isoxazolines, or combinations thereof.

Anthelmintic benzimidazoles comprise for example mebendazole, flubendazole, fenbendazole, oxfendazole, oxibendazole, albendazole, albendazole sulfoxide, thiabendazole, thiophanate, febantel, netobimin, and triclabendazole. Further examples include mebendazole, and ricobendazole.

Without wishing to be bound by theory, the inventors believe that benzimidazole-based anthelmintics may interfere with the worm's energy metabolism on a cellular level by binding to a specific building block called beta tubulin and preventing its incorporation into certain cellular structures called microtubules, which are essential for energy metabolism.

Imidazothiazoles and tetrahydropyrimidines are both nicotinic agonists. In some embodiments the one or more anthelmintic agents in at least one matrix may comprise imidathiazoles, for example levamisole, tetramisole, and butamisole. Tetrahydropyrimidine anthelmintics that may be used in the matrices of the invention include, for example, morantel, oxantel, and pyrantel.

Without wishing to be bound by theory the inventors believe that tetrahydropyrimidines may mimic the activity of acetylcholine, a naturally occurring neurotransmitter that initiates muscular contraction. This may lead to helminths that are unable to feed and starve.

Without wishing to be bound by theory the inventors believe that imidazothiazoles may have a similar mode of action to tetrahydropyrimidines and may cause spastic paralysis of helminths, For example, levamisole is thought to have a broad spectrum of activity and may therefore be effective against many larval stages of parasites.

In various embodiments the at least one matrix may comprise one or more macrocyclic lactones, for example abamectin, doramectin, eprinomectin, ivermectin, selamectin, milbemycin, for example as milbemycin oxime, moxidectin or a combination thereof.

In some embodiments the at least one matrix may comprise one or more salicylanilides for example brotianide, clioxanide, closantel, niclosamide, oxyclozanide, rafoxanide, substituted phenols including for example bithionol, disophenol, hexachlorophene, niclofolan, menichlopholan, nitroxynil, and aromatic amides, including for example diamfenetide (diamphenethide) or combinations thereof.

In some embodiments the at least one matrix may comprise one or more isoquinoline anthelmintics, such as for example praziquantel and epsiprantel. In some embodiments the matrices of the invention and the intra-ruminal devices may comprise one or more amino-acetonitrile derivatives, such as for example monepantel.

In some embodiments the at least one matrix may comprise one or more active ingredients such as for example piperazine and derivatives thereof such as piperazine and diethylcarbamazine (DEC, a derivative of piperazine), benzenesulfonamides such as clorsulon, amidines such as bunamidine, isothiocyantes such as nitroscanate, and organophosphates such as dichlorvos, and spiroindoles such as derquantel (2-deoxoparaherquamide).

In various embodiments, the one or more active ingredient(s) in the at least one matrix of the intra-ruminal device, is/are stable and do not react with other components in the at least one matrix or degrade or decompose by other means.

In various embodiments, the payout rates of the active ingredient(s) may be measured as a function of the width of a matrix ejected into the rumen through the one or more outlets in the end cap. In some embodiments the payout rate of the intra-ruminal device of the invention may be from about 0.1, 0.125, 0.15, 0.175, 0.2, 0.225, 0.025, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, or 1.2 mm or more per day in an aqueous medium, for example ruminal fluid or water, and suitable ranges may be selected from any of these values, for example from about 0.1 to about 1.2, about 0.1 to about 1, about 0.1 to about 0.75, 0.1 to about 0.6, 0.1 to about 0.5, 0.2 to about 1.2, about 0.2 to about 0.75, about 0.2 to about 0.6, about 0.2 to about 0.5 mm/day). It will be understood by a person skilled in the art that the payout rate as a function of the width of a matrix may depend on the size of the intra-ruminal device.

Preferably, the payout of the one or more active ingredients is linear or substantially linear. In various embodiments the linearity may be greater than from about 0.95, 0.955, 0.96, 0.965, 0.97, 0.975, 0.98, 0.985, 0.99, 0.995, 0.996, 0.997, 0.998, 0.999 or more and suitable ranges may be selected from any of these values, for example from about 0.95 to about 0.999, from about 0.99 to about 0.995, from about 0.99 to about 0.996, from about 0.99 to about 0.997, from about 0.99 to about 0.998, from about 0.99 to about 0.999.

In various embodiments, the payout rates of the one or more active ingredient(s) is/are minimally affected, preferably not affected by the pH and ionic composition of the rumen.

In some embodiments, the at least one matrix of the intra-ruminal device, may comprise more than one active ingredient. For example in some embodiments the matrices of the invention may comprise from 2, 3, 4, 5, 7, 8, 9, or about 10, or more active ingredients, and useful ranges may be selected from any of these values (for example from 2 to about 10 or from 2 to about 5 active ingredients).

In some embodiments the at least one matrix may comprise more than one active ingredient, wherein some or all of the active ingredients belong to a different therapeutic class, for example antibiotics, antifungals, antivirals, steroid hormones, antihistamines, metabolic regulators, productivity regulators, corticosteroids, anti-thyroidal agents, parasiticidal agents, such as for example anthelmintics and/or nutritional actives. For example the matrix may comprise 3 actives, one of which is an anthelmintic, one of which is an antibiotic and the third being a nutritional active, for example a vitamin.

In various embodiments the at least one matrix of the intra-ruminal device may comprise more than one active ingredient, each of which belongs to the same therapeutic class, preferably anthelminitics. In some embodiments the matrix may comprise two or more anthelmintic actives belonging to the same class of anthelmintics, such as for example benzimidazoles, imidazothiazoles, tetrahydropyrimidines, macrocyclic lactones, salicylanilides, substituted phenols, aromatic amides, isoquinolines, amino acetonitriles and spiroindoles. For example the at least one matrix may comprise two or three actives, each of which may be a macrocyclic lactone.

In various embodiments the at least one matrix of the intra-ruminal device may comprise two or more active ingredients each of which is an anthelmintic active and each belonging to a different anthelmintic class, such as for example benzimidazoles, imidazothiazoles, tetrahydropyrimidines, macrocyclic lactones, salicylanilides, substituted phenols, aromatic amides, isoquinolines, amino acetonitriles and spiroindoles. For example the matrices may comprise two anthelmintics, one of which may be a macrocyclic lactone and the other may be an imidazothiazole.

In some embodiments the at least one matrix may comprise at least about 5, 7.5, 10, 12.5, 15, 17.5, 20, 22.5, 25, 27.5, 30, 32.5, 35, 37.5, 40, 42.5, 45, 47.5, 50, 52.5 or 55% or more of one or more active ingredients by weight of each matrix, and useful ranges may be selected from any of these values (for example from about 5 to about 55, about 5 to about 50, about 5 to about 25, about 5 to about 10, about 6 to about 55, about 6 to about 50, about 6 to about 25, about 6 to about 10, about 7 to about 55, about 7 to about 50, about 7 to about 35, about 7 to about 10, about 8 to about 55, about 8 to about 50% about 8 to about 50, about 8 to about 25, about 8 to about 10, about 9 to about 55, about 9 to about 50, about 9 to about 25, about 10 to about 55, about 10 to about 50, about 10 to about 40, about 10 to about 30, about 10 to about 25, or about 10 to about 25% by weight of the matrix).

In various embodiments the matrix (tablet(s)) of the invention and the intra-ruminal devices comprising these tablets comprise a ratio of one or more active ingredients, polymers as described herein and other ingredients that allows for the delivery of a therapeutically effective amount of the one or more active ingredients to the non-human animal, preferably ruminant.

### 2.3. Other Ingredients

The at least one matrix comprising the one or more active ingredients and polymers may further comprise a number of excipients. Examples of suitable excipient may include, but are not limited to fillers, diluents, lubricants, surfactants, glidants, gel formers, binders, and stabilisers, or combinations thereof.

In some embodiments, the at least one matrix of the invention may further comprise one or more fillers or diluents. Examples of suitable fillers or diluents may include, but are not limited to, sugars such as for example lactose, sucrose and mannitol, inorganic salts such as calcium phosphate and calcium carbonate, cellulose, methyl cellulose, ethyl cellulose, aluminium silicates, kaolin or combinations thereof.

In some embodiments the at least one matrix may comprise one or more fillers and/or diluents at amounts of from about 0, 0.1, 0.25, 0.5, 0.75, 1, 2, 3, 4, 5, 6, 7, 8, 9, 10, 12.5, 15, 17.5, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95% by weight of the matrix, and useful ranges may be selected from any of these values (for example from about 0.1 to about 95, 0.1 to about 80, 0.1 to about 50, 0.1 to about 20, 0.1 to about 15, 0.1 to about 10, 0.1 to about 5, 5 to about 95, 5 to about 90, 5 to about 75, 5 to about 50, 5 to about 25, or about 5 to about 10% by weight of the matrix).

For example, in some embodiments the filler/diluent may comprise lactose and/or another filler such as for example sucrose or mannitol, or combinations thereof in an amount of about 0.1 to about 35% of the matrix.

In some embodiments the filler/diluent may comprise cellulose or a cellulose derivative such as for example, methyl cellulose and/or ethyl cellulose, or a combination of any two or more thereof, with or without the presence of one or more other fillers/diluents, in an amount of about 0.1 to about 80% by weight of the matrix.

In some embodiments the filler/diluent may comprise a filler/diluent selected from the group consisting of aluminium silicates, kaolin, calcium phosphate and calcium carbonate, or a combination of any two or more thereof, with or without the presence of one or more other fillers/diluents, in an amount of about 0.1 to about 80% by weight of the matrix.

In some embodiments, the at least one matrix may comprise one or more surfactants or lubricants. Examples of surfactants or lubricants may include, but are not limited to, stearates such as for example magnesium stearate calcium stearate, and stearyl fumarate, glyceryl stearates such as for example glyceryl monostearate, glycerine derivatives, sodium lauryl sulfate, sucrose fatty acid ester, poloxamer, mineral clays such as for example kaolin, aluminium silicates, or combinations thereof. In some embodiments the one or more surfactants and/or lubricants may be present in the matrices of the invention in an amount of from about 0.01, 0.05, 0.075, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, or 90% by weight of the matrix, and useful ranges may be selected from any of these values (for example from about 0.01 to about 90, about 0.01 to about 75, about 0.01 to about 50, about 0.01 to about 25, about 0.01 to about 10, about 0.5 to about 90, about 0.5 to about 75, about 0.5 to about 50, about 0.5 to about 25, about 5 to about 80, about 5 to about 60, about 5% to about 40, or about 5 to about 20%).

For example, in some embodiments the lubricant/surfactant may comprise stearates such as for example magnesium stearate or calcium stearate, stearyl fumarate, glyceryl stearates such as for example glyceryl monostearate, glycerine derivatives or combinations thereof, in an amount of about 0.05 to about 3% by weight of the matrix.

In some embodiments the lubricant/surfactant may comprise sodium lauryl sulfate in an amount of about 0.01 to about 5% by weight of the matrix.

In some embodiments the lubricant/surfactant may comprise one or more sucrose fatty acid esters in an amount of about 5 to about 80% by weight of the matrix.

In some embodiments the lubricant/surfactant may comprise one or more poloxamers in an amount of about 0.01 to about 10% by weight of the matrix.

In some embodiments the lubricant/surfactant may comprise one or more fillers such as one or more mineral clays and/or aluminium silicates, such as for example kaolin in an amount of about 0.1 to about 80% by weight of the matrix.

In some embodiments, the at least one matrix may further comprise one or more glidants. Examples of glidants include, but are not limited to, colloidal silicon dioxide, talc, metal stearates such as magnesium stearate, calcium stearate and stearyl fumarate, and glyceryl stearates such as glyceryl monostearate, or combinations thereof. In some embodiments the glidant(s) may be present in the at least one matrix in amounts of from about 0.01, 0.25, 0.5, 0.75, 1, 1.25, 1.5, 1.75, 2, 2.25, 2.5, 2.75, 3, 3.25, 3.5, 3.75, 4, 4.25, 4.5, 4.75, or 5% by weight of the matrix, and useful ranges may be selected from any of these values (for example from about 0.01 to about 5, about 0.01 to about 4, about 0.01 to about 2, about 0.01 to about 1, about 0.25 to about 5, about 0.25 to about 4, about 0.25 to about 3, about 0.25 to about 1, about 0.5 to about 5, about 0.5 to about 3, about 0.5 to about 2, about 0.5 to about 1% weight of the matrix).

In some embodiments the glidant may comprise colloidal silicon dioxide, talc, metal stearates such as magnesium stearate, calcium stearate and stearyl fumarate, and/or glyceryl stearates such as glyceryl monostearate, or combinations thereof in an amount of about 0.01 to about 2% by weight of the matrix.

In some embodiments, the at least one matrix may comprise one or more additional gel formers. Examples of additional gel formers that may be used include, but are not limited to, sucrose fatty acid ester, cellulosic derivatives such as hydroxyethyl cellulose and hydroxymethyl cellulose, and chitosan, or combinations thereof. The gel former(s) may be present in the at least one matrix in amounts of from about 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 55, 60, 65, 70, 75, 80, 85 or 90% by weight of the matrix, and useful ranges may be selected from any of these values (for example from about 0.1 to about 90, about 0.1 to about 80, about 0.1 to about 50, about 0.1 to about 20, about 0.1 to about 15, about 0.1 to about 10, about 0.5 to about 90, about 0.5 to about 80, about 0.5 to about 50, about 0.5 to about 30, about 0.1 to about 5, about 5 to about 90, about 5 to about 75, about 5 to about 50, about 5 to about 25, or about 5 to about 10% by weight of the matrix).

In some embodiments the gel former may comprise sucrose fatty acid ester in an amount of about 5 to about 80% by weight of the matrix.

In some embodiments the gel former may comprise one or more poly(ethylene) oxides in an amount of about 0.1 to about 90% by weight of the matrix.

In some embodiments the gel former may comprise one or more polyacrylic acid polymers, for example Carbomers, in an amount of about 0.01 to about 15% by weight of the matrix.

In some embodiments the gel former may comprise one or more cellulosic derivatives, for example hydroxyethyl cellulose and hydroxymethyl cellulose, or a combination thereof in an amount of about 0.01 to about 90% by weight of the matrix.

In some embodiments the gel former may comprise cellulose in an amount of about 0.01 to about 30% by weight of the matrix.

In some embodiments, the at least one matrix may comprise one or more binders. Examples of binders include, but are not limited to cellulosic derivatives such as hydroxyethyl cellulose and hydroxymethyl cellulose. The binder(s) may be present in the at least one matrix in amounts of from about 0, 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, or 50% by weight of the matrix, and useful ranges may be selected from any of these values (for example from about 0.1 to about 50, about 0.1 to about 35, about 0.1 to about 10, about 0.1 to about 10, about 0.5 to about 50, about 0.5 top about 25, about 0.5 to about 10, about 0.5 to about 5, about 1 to about 50, about 1 to about 35, about 1 to about 20, about 1 to about 10, or about 1 to about 5% by weight of the matrix).

In some embodiments the binder may comprise polyvinylpyrrolidone in an amount of about 0.01 to about 10% by weight of the matrix.

In some embodiments the binder may comprise one or more cellulosic derivatives, for example methyl and/or ethyl cellulose, or a combination thereof in an amount of about 0.01 to about 35% by weight of the matrix.

In some embodiments, the at least one matrix may comprise one or more stabilisers. Examples of stabilisers that may be used in the matrices include, but are not limited to, antioxidants such as for example butylated hydroxytoluene, butylated hydroxyanisole and tocopherol, and/or buffers.

The stabilisers(s) may be present in the at least one matrix in amounts of from about 0.01, 0.02, 0.03, 0.04, 0.05, 0.06, 0.07, 0.08, 0.09, 0.1, 0.15, 0.2, 0.25, 0.5, 0.75, 1, 1.25, 1.5, 1.75, 2, 2.25, 2.5, 2.75, 3, 3.25, 3.5, 3.75, 4, 4.25, 4.5, 4.75, or 5% by weight of the matrix, and useful ranges may be selected from any of these values (for example from about 0.01 to about 5, about 0.01 to about 3, about 0.01 to about 1, about 0.01 to about 5, 0.1 to about 5%, about 0.5 to about 3.5% by weight of the matrix).

In some embodiments the stabiliser may comprise one or more chemical stabilizers. For example, in some embodiments the stabiliser may comprise one or more antioxidants such as for example butylated hydroxytoluene, butylated hydroxyanisole and tocopherol, or combinations thereof in an amount of about 0.01 to about 10% by weight of the matrix.

In some embodiments the stabiliser may comprise one or more buffers in an amount of 0.1 to about 5% by weight of the matrix. Suitable buffers will be known to a person skilled in the art.

In some embodiments the at least one matrix may comprise lactose, magnesium stearate and a sucrose fatty acid ester as excipients.

In some embodiments the one or more matrix matrices comprise lactose in an amount of from about 0.1 to about 35%, magnesium stearate in an amount of from about 0.05 to about 3.0% and sucrose fatty acid ester in an amount of from about 5 to 80%.

In some embodiments the at least one matrix may comprise lactose, magnesium stearate, a sucrose fatty acid ester and colloidal silicon dioxide as excipients.

In some embodiments the at least one matrix may comprise lactose in an amount of from about 0.1 to about 35%, magnesium stearate in an amount of from about 0.05 to about 3.0% and sucrose fatty acid ester in an amount of from about 5 to 80% and colloidal silicon dioxide in an amount of from about 0.01 to about 2.0% by weight of the matrix.

### 3. Method of Manufacture

In some embodiments the present invention provides a method of manufacturing an intra-ruminal device as described herein.

In some embodiments the method comprises
granulating a mixture comprising at least one active ingredient, and at least one polymer selected from the group consisting of a non-ionic polymer and a cross-linked anionic polymers, and optionally one or more excipients as described herein,
drying the granules,
passing the granules through a sieve, and
tabletting/compressing the granules into at least one matrix, and
loading the at least one matrix into the body of an intra-ruminal device.

The granulated mixture may be prepared by wet or dry granulation and it will be apparent to a person skilled in the art that a number of granulation processes may be used. For example, the mixture may be prepared by wet granulation using a high-shear granulator, a fluidized bed granulator or by any other suitable means known to a person skilled in the art. In some embodiments the mixture may be granulated in a fluid-bed drier, for example by wet granulation comprising spraying a pharmaceutically acceptable solvent, for example water or a suitable alcohol or glycol ether onto the material to be granulated.

It will be understood by a person skilled in the art that pre-tabletting/compression processes other than fluid-bed granulation may be used. For example direct blending or other wet or dry granulation processes may be used.

In some embodiments the at least one matrix may be manufactured using a fluid-bed granulation process prior to the tablet compression process. In some embodiments a single stroke or a rotary tablet press may be used.

In various embodiments the matrices may undergo granulation or blending prior to compression.

In some embodiments granulation may comprise high shear mixing and/or roller compaction.

The at least one matrix of the invention may be compressed as flat-faced compacts, which means the matrices do not have limited or no curvature or edge bevel. The flat-faced matrices formed in this way may allow a continuous stack of matrices to be formed when assembled in the intra-ruminal device.

In some embodiments, the processing parameters such as air velocity, atomising air pressure and/or spray rate may be adjusted in order to provide granules of the desired attributes.

In some embodiments the air velocity used for granulation may be from at least about 2, 3, 4, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 Pa or more, and useful ranges may be selected from any of these values (for example from about 2 to about 24, about 2 to about 30, about 2 to about 28, about 2 to about 26, about 2 to about 24, about 2 to about 22, about 2 to about 20, about 2 to about 18, about 2 to about 16, about 2 to about 14, about 2 to about 12, about 2 to about 10, about 5 to about 45, about 5 to about 40, about 5 to about 20, about 5 to about 10 Pa).

In some embodiments the atomising air pressure may be from at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 bar or more, and useful ranges may be selected from any of these values (for example from about 0.5 to about 5.0, about 0.5 to about 2.5, about 0.5 to about 1.0, about 1.0 to about 5.0, about 1.0 to about 4, about 1.0 to about 3.0, about 1.0 to about 2.0, about 2.0 to about 5.0, or from about 2.0 to about 4.0 bar).

In some embodiments the spray rate is from at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 g/min or more, and useful ranges may be selected from any of these values (for example from about 5 to about 50, about 5 to about 30, about 5 to about 10, about 20 to about 50, about 20 to about 40, or about 20 to about 20 g/min).

In one embodiment the batches are dried at from about 20, 21, 22, 23, 24, 25, 26, 27, 28, 29, 30, 31, 32, 33, 34, 35, 36, 37, 38, 39, 40, 41, 42, 43, 44, or 45° C., and suitable ranges may be selected from any of these values (for example from about 20 to about 45, about 20 to about 30, about 25 to about 45, about 25 to about 35, about 30 to about 45 or about 30° C. to about 35° C.).

In some embodiments the batches are dried for at least about 0.5, 1, 2, 3, 4, 5, 6, 7, 8, 9, 10, 12, 14, 16, 18, 20, 22, 24, 30, 36, 42 or 48 hours or more, and suitable ranges may be selected from any of these values (for example at least about 0.5 to 48, about 0.5 to about 24, about 0.5 to about 12, about 0.5 to about 6, about 1 to about 48, about 1 to about 24, about 1 to about 12, about 1 to about 6, or about 1 to about 5 hours).

In some embodiments the batches are dried to a defined granule moisture level, for example batches may be dried until a loss on drying (LoD) value of at least about 1, 1.25, 1.5, 1.75, 2, 2.25, 2.5, 2.75, 3, 3.25, 3.50, 3.75, 4, 4.25, 4.5, 4.75, or 5% weight by weight (w/w) is achieved, and useful ranges may be selected from any of these values, for example from about 1 to about 5, about 1 to about 3, about 1.5 to about 5, about 1.5 to about 4, about 1.5 to about 3% w/w.

In some embodiment the dried granules are passed through a sieve, for example a 14 mesh sieve.

The above parameter ranges will apply when a Glatt GPCG 1 fluid bed drier is used. It will be understood by a person skilled in the art that a number of other machinery may be used and that the machinery used will affect the processing parameters described above. It will also be apparent to a person skilled in the art that the above fluid bed drier may be used for small-scale manufacture only. Methods for scaling up the granulation processes including suitable machinery will be apparent to a person skilled in the art.

The resulting granules are then tableted, for example using any suitable tablet press. In some embodiments the granules may be tableted using a single stroke press or a rotary tablet press.

In various embodiments the matrices are packaged for use in an intra-ruminal device.

In some embodiments at least one matrix are loaded in to an intra-ruminal device. In some embodiments the at least one matrix may be loaded into an intra-ruminal device manually or the loading step may be automated and performed by one or more machines.

### 4. Use of the Composition

The at least one matrix and intra-ruminal device when used together are capable of delivering a therapeutically effective amount of a range of active ingredients, such as for example anthelmintics, to non-human animals, preferably ruminants. The intra-ruminal device may deliver the active to the rumen by diffusion through the at least one outlet in one end of the intra-ruminal device.

In various embodiments the intra-ruminal device comprising the one or more active ingredients are used for treating an animal in need thereof. The suitability of the intra-ruminal device of the invention for treating a particular disease or condition, depends for example on the active ingredients present in the composition.

In various embodiments the intra-ruminal device comprising the one or more active ingredients are used to improve productivity, for example by improving growth and protein yield.

In various embodiments the intra-ruminal device comprising the one or more active ingredients are used to minimise the impact of a production animal, for example a ruminant, on the environment, for example by reducing greenhouse gas emissions and/or nitrates.

The term “treatment”, and related terms, such as “treating” and “treat” as used herein, relates generally to treatment, of a non-human animal, to achieve one or more desired therapeutic effects. The therapeutic effect may be, for example, the inhibition of progress of a disease or condition, including a reduction in the rate of progress, a halt in the rate of progress, amelioration, and/or cure. Treatment as a prophylactic measure is also contemplated. Treatment may comprise combination treatments and therapies, in which two or more treatments or therapies are used, for example, sequentially or simultaneously, in combination.

In various embodiments the present invention provides a method of treating a non-human animal, preferably a ruminant, in need thereof, the method comprising administering a therapeutically effective amount of one or more active ingredients in the form of the at least one matrix in an intra-ruminal device as described herein.

A person skilled in the art will be able to readily determine the appropriate dosage required to treat an animal suffering from one or more conditions and/or to prevent one or more conditions. The dosage will depend upon the active ingredient(s) present in the composition and may also depend on the frequency of administration, the sex, age, weight and general condition of the animal treated, the nature and severity of the condition treated, any concomitant diseases to be treated, and any other factors which will be evident to those skilled in the art.

In some embodiments, the intra-ruminal device provides a sustained delivery of one or more nutritional and/or pharmaceutically active ingredients over an extended period of time. In some embodiments, the one or more active ingredients may be delivered over a payout period of about 20, 25, 30, 35, 40, 45, 50, 55, 60, 65, 70, 75, 80, 85, 90, 95, 100, 105, 110, 115, 120, 125, 130, 135, 140, 145, or 150 days, and useful ranges may be chosen from any of these values, for example from about 20 to about 150 days, or from about 40 to 100 days.

In some embodiments, the composition provides a sustained delivery of one or more nutritional or pharmaceutically active ingredient(s) over an extended period of time that is independent of pH and ionic effects.

Although the present invention and its advantages have been described in detail, it should be understood that various changes, substitutions and alterations can be made herein without departing from the spirit and scope of the invention as defined in the appended claims.

The present invention will be further illustrated in the following examples which are given for illustration purposes only and are not intended to limit the invention in any way.

### EXAMPLES

### Example 1—Manufacture of Polymer Containing Capsules

Lab-scale batches of trial matrices were manufactured containing
42% w/w active ingredient (monensin sodium),
44% w/w sucrose fatty acid ester,
1.5% w/w magnesium stearate,
0.5% w/w colloidal silicon dioxide, and
polymer and lactose monohydrate as specified in Table 1.

TABLE 1
Batch summary

| | Polymer content | Lactose monohydrate |
|---|---|---|
| Polymer | (% w/w) | (% w/w) |
| Poloxamer 407 micro | 3 | 9 |
| Povidone | 4 | 8 |
| (Kollidon 30) | 6 | 6 |
| Polyethylene oxide | 2 | 10 |
| (Polyox 1105) | 5 | 7 |
| Polyethylene oxide | 4 | 8 |
| (Polyox 301) | 6 | 6 |
| Polyethylene oxide | 2 | 10 |
| (Polyox 303) | 5 | 7 |
| HMPC K4M | 3 | 9 |
| | 6 | 6 |
| Carbomer 980NF | 2 | 10 |
| | 5 | 7 |
| Carbomer Ultrez 10 | 2 | 10 |
| | 5 | 7 |

The active ingredient, sucrose fatty acid ester, lactose and polymer were granulated in a fluid-bed drier (Glatt GPCG 1 Fluid-bed granulator) by spraying water onto the materials. Processing parameters such as air velocity, atomising air pressure and spray rate were adjusted in order to provide granules of the required attributes. Air velocity was between 5 and 40 Pa. Atomising air pressure was between 2.0 and 4.0 bar. Spray rate was between 20 and 40 g/min. The batches were dried to 34° C. and passed through a 14 mesh sieve.

Mean particle size diameter, weight range, thickness range and hardness range of the resulting matrices are summarised in Table 2.

In each case, the tablets were within the standard expected ranges showing that the tabletting method was successful.

TABLE 2
Tabletting results

| | | Mean | | | |
|---|---|---|---|---|---|
| | | particle | | | |
| | Polymer | size | | Thickness | Hardness |
| | content | diameter | Weight | range | range |
| Polymer | (% w/w) | (μm) | range (g) | (mm) | (kp) |
| Poloxamer | 3 | 289 | 3.834-3.916 | 7.53-7.69 | 25.2-28.5 |
| 407 micro | | | | | |
| Kollidon ® | 4 | 281 | 3.849-3.894 | 7.59-7.63 | 28.8-36.7 |
| 30 | 6 | 262 | 3.903-3.956 | 7.55-7.67 | 28.9-35.8 |
| Polyox ® | 2 | 315 | 3.861-3.927 | 7.63-7.69 | 25.4-27.3 |
| 1105 | 5 | 257 | 3.844-3.908 | 7.62-7.67 | 29.4-31.8 |
| Polyox ® | 4 | 251 | 3.871-3.924 | 7.59-7.66 | 26.0-33.9 |
| 301 | 6 | 277 | 3.886-3.905 | 7.59-7.66 | 27.4-30.7 |
| Polyox ® | 2 | 264 | 3.825-3.903 | 7.60-7.70 | 26.2-29.6 |
| 303 | 5 | 300 | 3.880-3.926 | 7.59-7.64 | 29.4-32.2 |
| HPMC K4M | 3 | 241 | 3.857-3.905 | 7.60-7.67 | 25.0-28.4 |
| | 6 | 260 | 3.797-3.901 | 7.50-7.60 | 28.6-36.5 |
| Carbomer ® | 2 | 273 | 3.868-3.891 | 7.56-7.66 | 29.1-30.5 |
| 980NF | 5 | 199 | 3.829-3.970 | 7.54-7.69 | 37.5-41.6 |
| Carbomer ® | 2 | 250 | 3.815-3.895 | 7.56-7.69 | 29.2-31.7 |
| Ultrez 10 | 5 | 291 | 3.858-3.911 | 7.64-7.71 | 34.5-36.7 |

### Example 2—In Vitro Capsule Testing

Formulations were evaluated using a custom-made 240 L stainless steel tank. This model tests the susceptibility of the formulations described herein to changes in the mineral concentrations and/or ionic interactions.

The closed tank is thermostatically controlled to 39° C. and equipped with a piston. The piston drives a brush which wipes the outlet of the capsules in order to mimic the physical abrasion to the tablet stack, which is expected to occur in vivo. Capsules are placed in stainless-steel housing units so that approximately 3 mm of the bristles from the brush passes in to the outlet each time the brush passes beneath the capsules (every 10 minutes). The piston speed is tailored so that the travel time across the tank is 12-15 seconds. The tank is equipped with a pump so that the medium is constantly recirculating throughout the study.

Capsule payout was calculated by measuring the distance from the front of the outlet to the top of the plunger using digital calipers. Each capsule was measured twice in this way by rotating the capsule through 180° and the mean value was used to calculate payout rates.

Capsules were tested in the tank using N=6 replicates. The batches were formulations containing secondary gel-forming excipients, two batches were used as comparator formulations (that is, formulations containing conventional gel formers) assembled in to capsules and one batch contained placebo matrices. Capsules were distributed evenly along and across the tank to take account of any variability due to capsule position. All capsules contained 12 matrices and were fitted with outlets as specified.

The capsules were initially run in potable water until linear profiles were established. Subsequently, the concentration of calcium in the tank was gradually increased every 3-4 days and the effect of capsule kinetics determined. Table 3 provides a summary of the trial information.

TABLE 3
In vitro trial summary.

| | | | Mean payout rate (N = 6) | | |
|---|---|---|---|---|---|
| | | | (mm/day) | | |
| | Content | Outlet | Water | | |
| Polymer | (% w/w) | (mm) | Day 0-3 | 3-7 | 7-10 |
| Carbomer ® 971 | 3.0 | 12 | 1.656 | 1.578 | 1.572 |
| (Control) | | | | | |
| Polyox ® 1105 | 2.0 | 12 | 1.114 | 1.267 | 1.286 |
| | 5.0 | 12 | 0.603 | 1.156 | 1.078 |
| Polyox ® 301 | 4.0 | 12 | 0.610 | 1.151 | 1.059 |
| | 6.0 | 12 | 0.481 | 1.001 | 0.951 |
| Polyox ® 303 | 2.0 | 13 | 1.145 | 1.296 | 1.376 |
| | 5.0 | 13 | 0.715 | 1.259 | 1.173 |
| HPMC K4M | 3.0 | 13 | 1.049 | 1.194 | 1.240 |
| | 6.0 | 13 | 0.502 | 0.928 | 0.865 |
| Carbomer ® 980NF | 2.0 | 12 | 0.766 | 1.304 | 1.249 |
| | 5.0 | 12 | 0.777 | 1.204 | 1.127 |
| Carbomer ® Ultrez 10 | 2.0 | 12 | 0.936 | 1.325 | 1.325 |
| | 5.0 | 12 | 1.119 | 1.489 | 1.547 |

Note: In the control the active was replaced with lactose and sucrose ester.

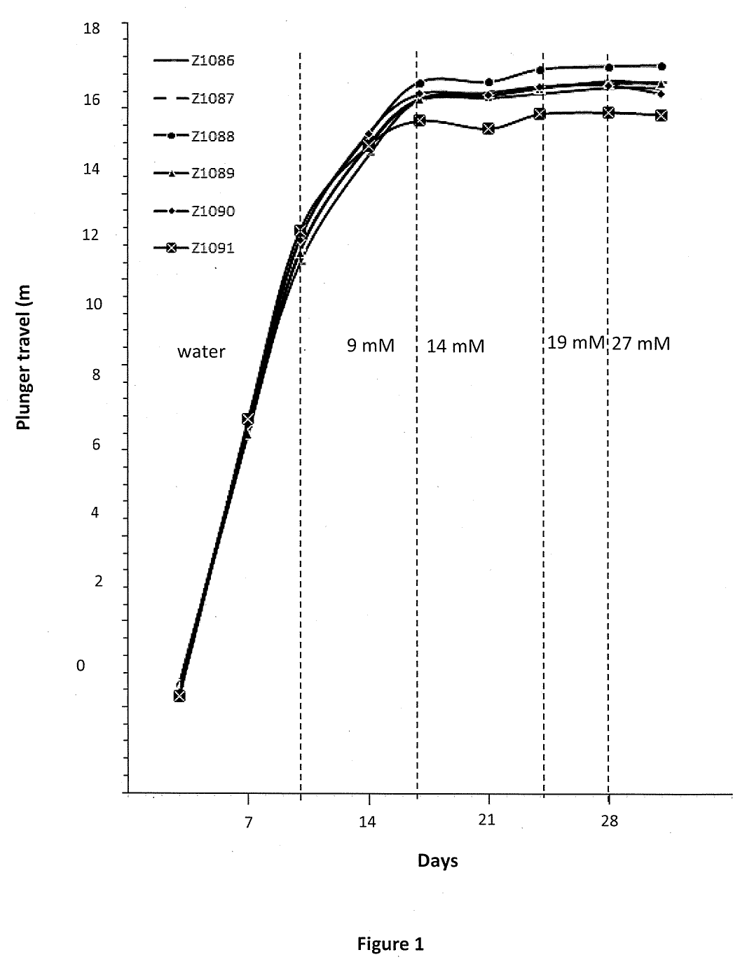

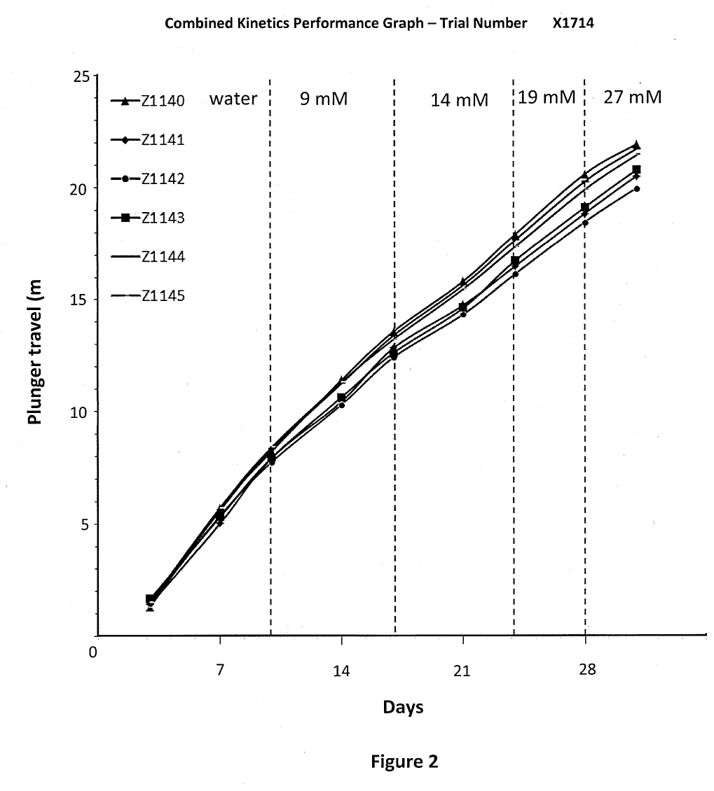

FIGS. 1 and 2 illustrate the payout profiles of the formulations of the Placebo (that is, the control formulation containing a cross-linked anionic polymer, Carbomer® 971) and the formulation containing Polyox 301 6% respectively. It can be seen that when these formulations are transferred from water to 9 mmol·L⁻¹ calcium at day 10 that there is a reduction in payout rate from these formulations. However, further increases in the calcium concentration have minimal effect on the payout rate from these formulations. Indeed, these formulations continue to payout at the highest calcium level studied.

TABLE 4
Trial summary.

| | | | Mean payout rate (N = 6) (mm/day) | | | | | |
|---|---|---|---|---|---|---|---|---|
| | | | Calcium acetate solution | | | | | |
| | Content | Outlet | 9 mM | | 14 mM | | 19 mM | 27 mM |
| Polymer | (% w/w) | (mm) | 10-14 | 14-17 | 17-21 | 21-24 | 24-28 | 28-31 |
| Carbomer ® | 3.0 | 12 | 1.402 | 0.915 | 1.146 | 0.845 | 1.007 | 0.854 |
| 971 (Control) | | | | | | | | |
| Polyox ® | 2.0 | 12 | 0.761 | 0.588 | 0.584 | 0.631 | 0.467 | 0.494 |
| 1105 | 5.0 | 12 | 0.785 | 0.566 | 0.488 | 0.656 | 0.564 | 0.347 |
| Polyox ® 301 | 4.0 | 12 | 0.794 | 0.578 | 0.435 | 0.611 | 0.513 | 0.386 |
| | 6.0 | 12 | 0.696 | 0.701 | 0.526 | 0.643 | 0.619 | 0.511 |
| Polyox ® 303 | 2.0 | 13 | 0.811 | 0.783 | 0.678 | 0.659 | 0.664 | 0.611 |
| | 5.0 | 13 | 0.894 | 0.730 | 0.737 | 0.822 | 0.640 | 0.686 |
| HMPC K4M | 3.0 | 13 | 0.646 | 0.524 | 0.326 | 0.538 | 0.495 | 0.380 |
| | 6.0 | 13 | 0.376 | 0.364 | 0.179 | 0.302 | 0.200 | 0.142 |
| Carbomer ® | 2.0 | 12 | 0.565 | 0.241 | 0.196 | 0.216 | 0.125 | 0.000 |
| 980NF | 5.0 | 12 | 0.743 | 0.570 | 0.512 | 0.604 | 0.299 | 0.184 |
| Carbomer ® | 2.0 | 12 | 0.568 | 0.315 | 0.153 | 0.174 | 0.063 | −0.020 |
| Ultrez 10 | 5.0 | 12 | 1.030 | 1.204 | 0.839 | 1.031 | 0.681 | 0.623 |

Note:
In the placebo the active was replaced with lactose and sucrose ester

The results show that the formulations comprising the polymers claimed ran in a linear fashion across a relatively wide range of calcium concentration. The most significant change in payout rate was observed when capsules were exposed to 9 mmol·L⁻¹ after being in water. However, further increases in calcium concentration did not markedly affect the payout rates. In vivo, there will always be calcium present in the rumen. Therefore, the payout reduction observed when water is changed to calcium may be an artefact which does not occur in vivo.

1. An intra-ruminal device comprising
a body substantially impervious to rumen fluid, the body comprising a barrel, at least one outlet, and at least one matrix in the barrel,
a compression arrangement within the body adapted to bias the column of matrices in the barrel to the at least one outlet, and
at least one variable geometry device, dependent from the body to assist rumen retention,
wherein the at least one matrix in the barrel comprises at least one active ingredient and at least one polymer selected from the group consisting of a non-ionic polymer and a cross-linked anionic polymer.

2. The intra-ruminal device of claim 1, wherein the non-ionic polymer is a homopolymer.

3. The intra-ruminal device of claim 1, wherein the non-ionic polymer is selected from the group consisting of polyethylene oxide polymers and polyvinylpyrrolidone.

4. The intra-ruminal device of any of claim 3, wherein the polyvinylpyrrolidone has a molecular weight ranging from 35,000 to 60,000 g/molL-1.

5. The intra-ruminal device of claim 3, wherein the polyethylene oxide polymer has a molecular weight ranging from 800,000 to 7,250,000 g/molL-1.

6. The intra-ruminal device of claim 5, wherein the polyethylene oxide polymer has a viscosity in the range 5,000 to 15,000 cP.

7. The intra-ruminal device of claim 1, wherein the cross-linked anionic polymer comprises less than 65% monomers with free anionic groups compared to the total number of monomers.

8. The intra-ruminal device of claim 1, wherein the cross-linked anionic polymer is a polyacrylic acid or a polyacrylic acid derivative.

9. The intra-ruminal device of claim 1, wherein the cross-linked anionic polymers are cross-linked with allyl sucrose or allylpentaerythritol.

10. The intra-ruminal device of claim 1, wherein the cross-linked anionic polymers have a viscosity in the range 40,000 to 67,000 Cp.

11. The intra-ruminal device of claim 1, wherein the cross-linked anionic polymer comprises a block copolymer of polyethylene glycol and a long chain alkyl acid ester.

12.-14. (canceled)

15. The intra-ruminal device of claim 1, wherein the at least one active or beneficial ingredient is selected from the group consisting of antibiotics, antifungals, antivirals, steroid hormones, antihistamines, metabolic regulators, productivity regulators, corticosteroids, anti-thyroidal agents, and parasiticides.

16. The intra-ruminal device of claim 1, wherein the at least one active ingredient is a parasiticide

17. The intra-ruminal device of claim 16, wherein the parasiticide is an anthelmintic selected from the group consisting of benzimidazoles, imidazothiazoles, tetrahydropyrimidines, macrocyclic lactones, salicylanides, substituted phenols, aromatic amides, isoquinolines, amino acetonitriles amd spiroindoles, or a combination thereof.

18. A method of treating a ruminant animal in need thereof the method comprising administering the intra-ruminal device of claim 1 to the ruminant animal.

19. The method of claim 18, wherein the method of treating is independent of pH and ionic effects in the rumen of the ruminant animal.

20. A method of treating a ruminant animal in need thereof, the method comprising administering the intra-ruminal device of claim 1 to the ruminant animal, wherein the method of treating is independent of pH and ionic effects resulting from changes in the diet of the ruminant animal.

21.-24. (canceled)

25. A method of assembling a controlled delivery intra-ruminal device claim 1, the method comprising:
granulating a mixture comprising at least one active ingredient, at least one polymer selected from the group consisting of a non-ionic polymer and a cross-linked anionic polymers, and optionally one or more excipients,
drying the granules,
passing the granules through a sieve,
tableting the granules into at least one matrix, and
loading the at least one matrix into the body of an intra-ruminal device.

26. (canceled)

27. The intra-ruminal device of claim 16, wherein the parasiticide is an ectoparasiticidal agent and/or an endoparasiticidal agent.

28. The intra-ruminal device of claim 16, wherein the parasiticide is selected from the group consisting of anthelmintics, non-steroidal anti-inflammatories, nutritional actives, ruminal fermentation modifiers, or a combination thereof.
